(19) United States
(12) Patent Application Publication
(10) Pub. No.: US 2005/0214224 A1
(43) Pub. Date: Sep. 29, 2005

(54) Lipid formulations for spontaneous drug encapsulation

(75) Inventors: Jeffry G. Weers, Princeton Jct., NJ (US); Thomas Tarara, Burlingame, CA (US); Stelios Tzannis, Newark, CA (US)

Correspondence Address: NEKTAR THERAPEUTICS, 150 INDUSTRIAL ROAD, SAN CARLOS, CA, 94070, US

(73) Assignee: Nektar Therapeutics, San Carlos, CA (US)

(21) Appl. No.: 10/982,191

(22) Filed: Nov. 4, 2004

Related U.S. Application Data
(60) Provisional application No. 60/517,621, filed on Nov. 4, 2003.

(51) Int. Cl. A61L 9/04

(52) U.S. Cl. 424/45

(57) ABSTRACT

A pharmaceutical formulation for pulmonary administration is disclosed. The pharmaceutical formulation comprises a lipid component and an active agent, wherein the pharmaceutical formulation has a liquid phase transition temperature of less than or equal to 37° C. when hydrated and a liquid phase transition temperature of greater than 57° C. when non-hydrated, whereby the lipid component spontaneously encapsulates and/or entraps the active agent when the pharmaceutical formulation is administered to the lungs. A targeting agent may also be provided. In one version, the pharmaceutical formulation is useful to treat an infection, such as an inhalation anthrax infection.

This application claims the benefit U.S. Provisional Patent Application Ser. No. 60/571,621 filed on Nov. 4, 2003, which is incorporated herein by reference in its entirety.

### BACKGROUND

The need for effective therapeutic treatment of patients has resulted in the development of a variety of pharmaceutical formulation delivery techniques. One traditional technique involves the oral delivery of a pharmaceutical formulation in the form of a pill, capsule, elixir, or the like. However, oral delivery can in some cases be undesirable. For example, many pharmaceutical formulations may be degraded in the digestive tract before they can be effectively absorbed by the body. Inhaleable drug delivery, where an aerosolized pharmaceutical formulation is orally or nasally inhaled by a patient to deliver the formulation to the patient's respiratory tract, has proven to be a particularly effective and/or desirable alternative. For example, in one inhalation technique, an aerosolized pharmaceutical formulation provides local therapeutic relief to a portion of the respiratory tract, such as the lungs, to treat diseases such as asthma, emphysema, and cystic fibrosis. In another inhalation technique, a pharmaceutical formulation is delivered deep within a patient's lungs where it may be absorbed into the blood stream. Many types of aerosolization devices exist including devices that aerosolize a dry powder, devices comprising a pharmaceutical formulation stored in or with a propellant, devices which use a compressed gas to aerosolize a liquid pharmaceutical formulation, and similar devices.

Many forms of dry powder pharmaceutical formulations have been developed for administering an active agent to the lungs of a user. The powders are desirably composed of particles having a mean geometric diameter and/or a mass median diameter less than 20 μm and a mean aerodynamic diameter and/or a mass median aerodynamic diameter less than 5 μm to allow the pharmaceutical formulation to reach the deep lung. However, it is sometimes difficult to achieve the desired aerosol characteristics with dry powder formulations because the pharmaceutical powders often will agglomerate or otherwise have flow control difficulties making the powders difficult to administer effectively and consistently. In addition, when the powders have poor flow properties, the powders can be difficult to process and fill into receptacles. The aerosol properties and/or the flow properties of dry powders can be improved by adding one or more excipients to the formulation. For example, some powder pharmaceutical formulations comprise an active agent that is incorporated into a lipid and/or a polymer matrix.

Although the lipid and/or polymer in the pharmaceutical formulation can be tailored to improve the lung delivery characteristics of particular active agents and to improve its flow properties, it is often difficult to control the amount of time the active agent is retained within the lungs and/or to control the effectiveness of the active agent. Attempts have been made to increase the lung retention of an active agent in the lungs by encapsulating the active agent, such as by entrapping the active agent within the phospholipid bilayers of a spherical liposome. However, the production and stability of these liposomes has proven to be problematic. For example, liposome suspensions have been shown to disrupt during aerosolization via conventional nebulizers thereby releasing active drug prematurely. Additionally, active drug will often leak from liquid liposomes during prolonged storage. Dry lioposomes also have functional and stabilities issues as often the liposomes will burst during lyophilization, milling, or during reconstitution resulting in the leakage of active agent. In addition, powder liposomes tend to highly agglomerate and require deagglomeration before administration. This deagglomeration can be hard to control and can lead to additional liposome bursting with more leakage of the active agent.

Attempts have been made to develop pro-liposome powders where an active agent is spontaneously encapsulated when reconstituted with water. However, such pro-liposomes have historically been unstable and/or have suffered from an inability to adequately and consistently be delivered to the deep lungs of a user.

Therefore, it is desirable to provide a dry powder pharmaceutical formulation that spontaneously encapsulates an active agent when delivered to the lungs of a user. It is further desirable for the dry powder pharmaceutical formulation to be stable during storage and to be efficiently and reproducibly delivered to the lungs of the user.

### SUMMARY

The present invention satisfies these needs. In one aspect of the invention, a pharmaceutical formulation for pulmonary administration comprises a lipid component, and an active agent, wherein the pharmaceutical formulation has a liquid phase transition temperature of less than or equal to 37° C. when hydrated and a liquid phase transition temperature of greater than 57° C. when non-hydrated, whereby the lipid component spontaneously encapsulates and/or entraps the active agent when the pharmaceutical formulation is administered to the lungs. 10 In another aspect of the invention, a pharmaceutical formulation for pulmonary administration comprises a lipid component; an active agent; and divalent cations, wherein the pharmaceutical formulation has a liquid phase transition temperature of less than or equal to 37° C. when hydrated and wherein the divalent cations are present in an amount sufficient for the pharmaceutical formulation to have a liquid phase transition temperature of greater than 57° C. when non-hydrated.

In another aspect of the invention, a pharmaceutical formulation for pulmonary administration comprises a lipid component and ciprofloxacin, wherein the pharmaceutical formulation has a liquid phase transition temperature of less than or equal to 37° C. when hydrated, whereby the lipid component spontaneously encapsulates and/or entraps the active agent when the pharmaceutical formulation is administered to the lungs.

In another aspect of the invention, a pharmaceutical formulation for pulmonary administration comprises a lipid component; an active agent; and a targeting agent, wherein the pharmaceutical formulation has a liquid phase transition temperature of less than or equal to 37° C. when hydrated, whereby the lipid component spontaneously encapsulates and/or entraps the active agent when the pharmaceutical formulation is administered to the lungs.

In another aspect of the invention, a method of administering an active agent to the lungs of a user comprises: providing a receptacle containing a dry powder pharmaceutical formulation comprising a lipid component and an active agent; aerosolizing the pharmaceutical formulation and administering the pharmaceutical formulation to the lungs of the user during the user's inhalation; and spontaneously encapsulating or entrapping the active agent within the lipid when the pharmaceutical formulation enters the lungs.

In another aspect of the invention, a method of treating exposure to inhalation anthrax comprises providing a receptacle containing a dry powder pharmaceutical formulation comprising a lipid component and ciprofloxacin; aerosolizing at least 5 mg of the pharmaceutical formulation and administering the at least 5 mg of the pharmaceutical formulation to lungs of a user exposed to the inhalation anthrax; and spontaneously encapsulating the ciprofloxacin within the lipid when the pharmaceutical formulation enters the lungs.

In another aspect of the invention, a method of preventing inhalation anthrax infection comprises providing a receptacle containing a pharmaceutical formulation comprising a lipid component and ciprofloxacin; aerosolizing at least 5 mg of the pharmaceutical formulation and administering the at least 5 mg of the pharmaceutical formulation to lungs of a user that may become exposed to inhalation anthrax; and spontaneously encapsulating the ciprofloxacin within the lipid when the pharmaceutical formulation enters the lungs.

### DRAWINGS

These features, aspects, and advantages of the present invention will become better understood with regard to the following description, appended claims, and accompanying drawings which illustrate exemplary features of the invention. However, it is to be understood that each of the features can be used in the invention in general, not merely in the context of the particular drawings, and the invention includes any combination of these features, where:

FIG. 1 is a schematic representation of a pharmaceutical formulation administration according to the present invention, and

FIGS. 2A through 2E are schematic sectional side views showing the operation of an aerosolization apparatus delivering a pharmaceutical formulation according to the present invention.

### DESCRIPTION

The present invention relates to an aerosolizable pharmaceutical formulation. In particular, the invention relates to an aerosolizable dry powder pharmaceutical formulation comprising a lipid component and an active agent. Although the invention is illustrated in the context of a dry powder formulation wherein the active agent is spontaneously encapsulated when administered to the lungs of a user aerosolizing a dry powder pharmaceutical formulation for inhalation, the present invention can be used in other processes and should not be limited to the examples provided herein.

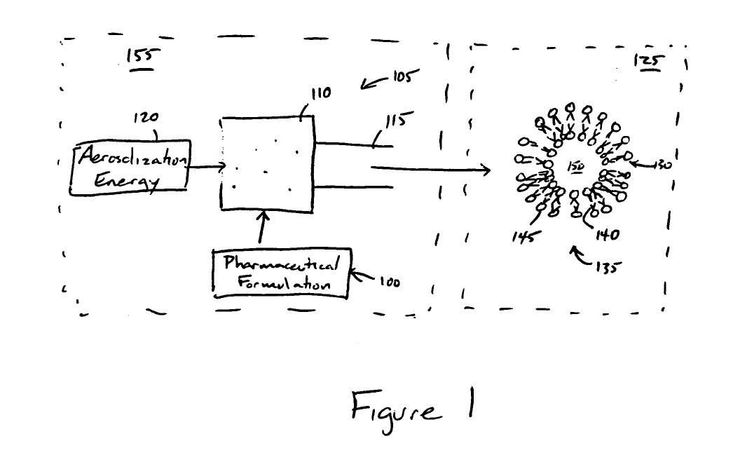

A pharmaceutical formulation according to the invention is shown in FIG. 1. The pharmaceutical formulation 100 comprises a lipid component and an active agent. The lipid component may comprise a single lipid or mixture of two or more lipids, and optionally any additional materials. An aerosolization device 105 is provided which comprises an aerosolization chamber 110 and a mouthpiece 115, which may alternatively be a nose piece or other opening. The pharmaceutical formulation 100 is introduced into the aerosolization chamber 110 and aerosolization energy 120 is provided to aerosolize the pharmaceutical formulation 100 so that it may be delivered through the mouthpiece 115 to a user's respiratory tract.

The aerosolized pharmaceutical formulation is introduced into a user's lungs 125, or other hydrated environment 125, in a manner where the active agent can be effectively administered. The pharmaceutical formulation has a gel (or solid) to liquid phase transition temperature in the hydrated state that is sufficiently low that the pharmaceutical formulation is in a liquid state after it is deposited in the fluid lining of the lungs. In order for the pharmaceutical formulation to be in a liquid state in the lungs of a human, the pharmaceutical formulation will have a gel (or solid) to liquid phase transition temperature in the hydrated state of less than or equal to 37° C. By being in the liquid state, the lipid molecules 130 in the lipid component of the pharmaceutical formulation spontaneously reassemble to form liposomes 135. These spontaneously formed liposomes 135 are lipid vesicles formed by the creation of one ore more lipid bilayers where the non-polar ends 140 of the lipid molecules 130 are oriented towards one another and the polar ends 145 are oriented away from one another. This creates an encapsulated vesicle where hydrophilic substances may be contained within the interior 150 of the liposome 130 or between bilayers in a multilamellar vesicle, and where lipophilic substances may be contained within the lipid bilayers and/or within pockets within the lipid bilayers.

The encapsulation of active agent within the liposomes increases the amount of time the active agent is retained in the lungs. This is due to liposome encapsulating the active agent in a manner that extends the half life of the active agent in the lungs beyond the active agent's half life when not encapsulated in a liposome. In one version, the active agent is encapsulated in a manner whereby the active agent has a half life in the lungs of at least 1 hr, in some cases at least 3 hours, in some cases at least 6 hours, in some cases at least 12 hours, and in some cases at least 24 hours. The desired half life of the active agent depends on the active agent, the dosage of the active agent, the desired activity of the active agent, and the types of lipids in the formulation and their proportions. In addition, the relative proportions of lipid component and active agent can be adjusted in order for a desirable amount of the active agent to become encapsulated. For example, in one version, at least 50% of the active agent is spontaneously encapsulated. In another version, at least 70% of the active agent in spontaneously encapsulated. This degree of encapsulation may be provided so that an active agent may have some immediate activity and some sustained activity.

In one version, the pharmaceutical formulation undergoes a transition into the liquid state once it is delivered to the lungs 125, or other hydrated environment. For example, the pharmaceutical formulation may be in a solid or gel form when in an ambient environment 155 and then transition into its liquid state when in a hydrated environment, such as the lungs 125. One way to alter the state of the pharmaceutical formulation is to tailor the pharmaceutical formulation so that its liquid transition temperature is between the temperature of the ambient environment and the temperature of the lungs, which is approximately 37° C. for a human. In that way, the pharmaceutical formulation may be in a solid or gel state when provided to the aerosolization device 105 and may transition to its liquid state when administered to the lungs.

The stability of the pharmaceutical formulation 100 can be improved by increasing its liquid transition temperature in the ambient environment 155. Accordingly, in one version, the pharmaceutical formulation has a first liquid transition temperature in a non-hydrated environment and a second liquid transition temperature in the lungs 125, or other hydrated environment. For example, in one version, the non-hydrated liquid transition temperature is higher than the hydrated liquid transition temperature. Thus, for a pharmaceutical formulation that is to be administered to the lungs of a human, the pharmaceutical formulation according to the present invention has a hydrated liquid transition temperature less than or equal to 37° C. and a non-hydrated liquid transition temperature greater than 37° C. By making the non-hydrated liquid transition temperature higher, the pharmaceutical formulation can be used in an ambient environment with less need for refrigeration and can be stored at room temperature for a longer period of time. By hydrated environment it is meant an environment having at least 90% relative humidity. By non-hydrated environment, it is meant an environment having less than 30% relative humidity. The storage stability of the pharmaceutical formulation can be further enhanced by increasing the difference between the hydrated liquid transition temperature of the pharmaceutical formulation and the non-hydrated liquid transition temperature. For example, in one version, the non-hydrated liquid transition temperature is at least 20° C. higher than the hydrated liquid transition temperature, more preferably at least 30° C. higher, and most preferably at least 40° C. higher. Therefore, for a pharmaceutical formulation that is to be administered to the lungs of a human, the pharmaceutical formulation may have a hydrated liquid transition temperature less than or equal to 37° C. and a non-hydrated liquid transition temperature greater than 57° C., more preferably a hydrated liquid transition temperature less than or equal to 37° C. and a non-hydrated liquid transition temperature greater than 67° C., and most preferably a hydrated liquid transition temperature less than or equal to 37° C. and a non-hydrated liquid transition temperature greater than 77° C. The increased difference between the transition temperatures also improves the aerosol properties and flow properties of the pharmaceutical formulation in the ambient environment.

The transition temperature properties of the pharmaceutical formulation may be tailored to meet the characteristics described above by proper selection of the lipid component and/or by mixing lipid components. For example, in one version the lipid component may comprise one ore more phospholipids. Many phospholipids have a hydrated liquid crystal phase transition temperature less than or equal to 37° C., or other desirable temperature. Accordingly, in one version, the lipid component comprises a phospholipid having a hydrated liquid transition temperature less than 37° C., such as dimyristoylphosphatidylcholine (DMPC) which has a hydrated liquid transition temperature of 23.5° C. Alternatively or additionally, the lipid component may comprise one or more other phospholipids having a hydrated liquid transition temperature of less than 37° C., such as one or more of the following phospholipids: saturated phospholipids having an acyl chain length of C14:0, C13:0, C12:0, C11:0, and C10:0 of phosphatidylcholine, phosphatyidyl ethanolamine, phosphatidylserine, phosphatidylglycerol, phosphatic acid, and cardiolipin, and unsaturated phospholipids, such as dioleylphosphatidylcholine and natural unsaturated phospholipids, such as egg PC, and other phospholipids known in the art.

In another version, the lipid component of the pharmaceutical formulation may comprise a mixture of phospholipids in order to provide desirable transition temperature characteristics. For example, by increasing the lipid component's liquid transition temperature to a value that is closer to 37° C., the difference between the ambient temperature and the transition temperature is increased, the transition temperature is still sufficiently low to allow for the lipid component to become liquid when in the lungs of a user. Therefore, in one version, a phospholipid having a hydrated liquid transition temperature well below 37° C. is combined with a phospholipid having a hydrated liquid transition temperature above 37° C. in a ratio that results in a phospholipid mixture having a hydrated liquid transition temperature that is 37° C. or is just below 37° C. For example, in one version DMPC is combined with dipalmitoylphosphatidylcholine (DPPC) which has a hydrated liquid transition temperature of 42° C. In another version, one or more of the above listed phospholipids having a hydrated liquid transition temperature below 37° C. is mixed with one or more of the following phospholipids having a hydrated liquid transition temperature above 37° C., such as one or more of the following phospholipids: saturated phospholipids having an acyl chainlength of C15:0, C16:0, C17:0, C18:0, C19:0, C20:0, C21:0, C22:0, C23:0, and C24:0 of phosphatidylcholine, phosphatyidyl ethanolamine, phosphatidylserine, phosphatidylglycerol, phosphatic acid, cardiolipin, and sphingolipids.

In another version, the lipid component of the pharmaceutical formulation may comprise a one or more phospholipids combined with one or more non-phospholipid lipids, such as sterols, fatty acids, and their salts. Examples of sterols include cholesterol, ergosterol, and the like. Examples of fatty acids include saturated and unsaturated lipids of chain length C12 to C20, such as myristic, palmitic, stearic, eicosanoic, acid and salts thereof. Inclusion of cholesterol in the lipid component will stabilize the phospholipids bilayers by inserting itself between neighboring lipid chains and thereby modifying the release of the entrapped active from the liposomal formulation.

In another version, the lipid component of the pharmaceutical formulation may comprise one or more non-ionic, non-phospholipid lipids that have a phase transition temperature less than or equal to 37° C. Non-phospholipid vesicles can be formed, for example, by mixtures of acid salts of quanternary amines, fatty alcohols and acids, fatty acid diethanolamines, ethoxylated fatty alcohols and acids, glycol esters of fatty acids, fatty acyl sarcocinates, glycerol fatty acid mono and diesters, ethoxylated glycerol fatty acid esters, glyceryl ethers and dimethyl amides.

The pharmaceutical formulation comprises a sufficient amount of the lipid component that the gel (or solid) to liquid phase transition temperature in the hydrated state is less than or equal to 37° C. This may be accomplished by having the lipid component itself have a gel (or solid) to liquid phase transition temperature in the hydrated state is less than or equal to 37° C. when the active agent and any additional materials in the pharmaceutical formulation do not effect the gel (or solid) to liquid phase transition temperature. Alternatively, the gel (or solid) to liquid phase transition temperature of the lipid component may be above or significantly below 37° C. in cases where the active agent and/or other materials alter the gel (or solid) to liquid phase transition temperature. In the latter case, the proportion of the lipids in the lipid component and the proportion of the lipid component in the pharmaceutical formulation may be tailored so that the gel (or solid) to liquid phase transition temperature of the pharmaceutical formulation is less than or equal to 37° C.

When the pharmaceutical formulation comprises an active agent for deliver to the lungs, the phospholipid content of the pharmaceutical formulation may be from 0.1% to 99.9%, preferably from 20% to 99.9%. The precise percentages are dependent on the active agent, the dose, the form of delivery, the desired degree of spontaneous encapsulation, and other factors. The active agent load accordingly. In one version, the phospholipid itself may be the active agent, such as when delivering natural or synthetic lung surfactant to the lungs.

In one version, the lipid component of the pharmaceutical formulation comprises one or more charged phospholipids. For example, the lipid component may comprise one of more of phosphatidylglycerols, phoshatidylserine, phosphatidylinositols, and PEGylated derivatives thereof. Electrostatic repulsion between charged headgroups increases interbilayer thicknesses, facilitating increases in solubilization capacity of the vesicular structures, thereby enabling higher drug loading and potentially increasing the encapsulation efficiency. The use of charged phospholipids may in some cases also facilitate increases in encapsulation and retention for oppositely charged active agents.

By adding one or more additives to the pharmaceutical formulation, the transition temperature properties of the pharmaceutical formulation may be further desirably affected. For example, the pharmaceutical formulation may comprise one or more phospholipids, as described above and may also comprise added salts that can impact the hydrated and/or the non-hydrated liquid transition temperature of the pharmaceutical formulation. For example, one or more polyvalent cations may be added to the pharmaceutical formulation to increase the non-hydrated liquid transition temperature. This increase in the non-hydrated liquid transition temperature increases the storage stability of the pharmaceutical formulation, reduces the impact of humidity on the pharmaceutical formulation, and allows for improved processing of the pharmaceutical formulation. Binding of divalent cations to the negatively charged phosphate group of zwitterionic phosphatidylcholines and phosphatidylethanolamines leads to lipids with anionic character. The addition of divalent cations is described in PCT publications WO 01/85136 and WO 01/85137, both of which are incorporated herein by reference in their entireties.

In one version, the pharmaceutical formulation comprises a polyvalent cation that is a divalent cation, such one or more of calcium, magnesium, zinc, iron, and the like. The polyvalent cation may be present in an amount effective to increase the non-hydrated liquid transition temperature of the phospholipid such that the particulate composition exhibits a liquid transition temperature which is greater than its storage temperature by at least 20° C., preferably at least 40° C. The molar ratio of polyvalent cation to phospholipid should be at least 0.05, preferably 0.05-2.0, and most preferably 0.25-1.0. A molar ratio of polyvalent cation:phospholipid of about 0.50 is particularly preferred according to the present invention. In one particular version the polyvalent cation is calcium. For example, the pharmaceutical formulation may comprise calcium chloride in an amount sufficient to increase the non-hydrated liquid transition temperature of the pharmaceutical formulation, such as by having a sufficient amount of calcium chloride to provide a molar ratio of calcium to phospholipid of at least 0.05, preferably of at least 0.25, and most preferably of at least 0.5.

In one particular version, the pharmaceutical formulation comprises a lipid component comprising a mixture of phospholipids and a polyvalent cation. For example, the lipid component may comprise a mixture of DMPC and DPPC in an amount sufficient to provide a hydrated liquid transition temperature of just below 37° C., and the pharmaceutical formulation may further comprise calcium chloride in a sufficient amount to raise the non-hydrated liquid transition temperature to at least 80° C., more preferably to at least 90° C. In one version, the lipid component may comprise from 20% to 50% DMPC and from 50% to 80% DPPC, and the calcium may be present in a molar ratio of calcium to phospholipid of about 0.5.

In one version, the pharmaceutical formulation may be delivered to the lungs of a user in the form of a dry powder. The dry powder pharmaceutical formulation becomes deposited in the lung's fluid lining where it transitions to the liquid state whereby liposomes may be formed to encapsulate the active agent, as described above. Accordingly, the pharmaceutical formulation comprises a dry powder that may be effectively delivered to the deep lungs or to another target site.

The pharmaceutical formulation according to this version of the invention is in the form of a dry powder which is composed of particles having a particle size selected to permit penetration into the alveoli of the lungs. Ideally for this delivery, the mass median aerodynamic diameter of the particles is less than 5 μm, and preferably less than 3 μm, and most preferably between 1 μm and 3 μm. The mass median diameter of the particles may be less than 20 μm, more preferably less than 10 μm, more preferably less than 6 μm, and most preferably from 2 μm to 4 μm. The delivered dose efficiency (DDE) of these powders may be greater than 30%, more preferably greater than 40%, more preferably greater than 50%, more preferably greater than 60%, and most preferably greater than 70%. These dry powders have a moisture content less than about 15% by weight, more preferably less than about 10% by weight, and most preferably less than about 5% by weight. Such powders are described in WO 95/24183, WO 96/32149, WO 99/16419, WO 99/16420, and WO 99/16422, all of which are all incorporated herein by reference in their entireties. “Mass median diameter” or “MMD” is a measure of mean particle size, since the powders of the invention are generally polydisperse (i.e., consist of a range of particle sizes). MMD values as reported herein are determined by centrifugal sedimentation and/or by laser diffraction, although any number of commonly employed techniques can be used for measuring mean particle size. “Mass median aerodynamic diameter” or “MMAD” is a measure of the aerodynamic size of a dispersed particle. The aerodynamic diameter is used to describe an aerosolized powder in terms of its settling behavior, and is the diameter of a unit density sphere having the same settling velocity, generally in air, as the particle. The aerodynamic diameter encompasses particle shape, density and physical size of a particle. As used herein, MMAD refers to the midpoint or median of the aerodynamic particle size distribution of an aerosolized powder determined by cascade impaction.

In one version, the pharmaceutical formulation comprises an active agent incorporated into a phospholipid matrix. By selecting the phospholipid component of the phospholipid matrix to have the liquid transition properties described above, the phospholipid component serves as both the matrix for transporting the active agent and also serves as the source of lipid molecules for the spontaneous encapsulation of the active agent in a liposome when the pharmaceutical formulation transitions to its liquid state. Examples of phospholipid matrices are described in WO 99/16419, WO 99/16420, WO 99/16422, WO 01/85136 and WO 01/85137 and in U.S. Pat. Nos. 5,874,064; 5,985,309; and 6,503,480, all of which are incorporated herein by reference in their entireties.

The pharmaceutical formulation may comprise phospholipid matrices that incorporate the active agent and that are in the form of particles that are hollow and/or porous microstructures, as described in the aforementioned in WO 99/16419, WO 99/16420, WO 99/16422, WO 01/85136 and WO 01/85137. The hollow and/or porous microstructures are particularly useful in delivering the active agent to the lungs because the density, size, and aerodynamic qualities of the hollow and/or porous microstructures are ideal for transport into the deep lungs during a user's inhalation. In addition, the phospholipid-based hollow and/or porous microstructures reduce the attraction forces between particles, making the pharmaceutical formulation easier to deagglomerate during aerosolization and improving the flow properties of the pharmaceutical formulation making it easier to process. The hollow and/or porous microstructures may exhibit, define or comprise voids, pores, defects, hollows, spaces, interstitial spaces, apertures, perforations or holes, and may be spherical, collapsed, deformed or fractured particulates.

In addition to the phospholipid and/or the polyvalent cation, an additive such as a co-surfactant or combinations of surfactants may be provided in the pharmaceutical formulation. For example, the hollow and/or porous microstructure may incorporate, adsorb, absorb, be coated with or be formed by the surfactant. The additional surfactant may comprise fluorinated and non-fluorinated compounds and are selected from the group consisting of saturated and unsaturated lipids, nonionic detergents, nonionic block copolymers, ionic surfactants and combinations thereof. Alternatively or additionally, an additive may comprise a compatible nonionic detergents suitable as co-surfactants, such as one ore more of: sorbitan esters including sorbitan trioleate, sorbitan sesquioleate, sorbitan monooleate, sorbitan monolaurate, polyoxyethylene, sorbitan monolaurate, and polyoxyethylene, sorbitan monooleate, oleyl polyoxyethylene ether, stearyl polyoxyethylene ether, lauryl polyoxyethylene ether, glycerol esters, and sucrose esters. The additive may be a block copolymer, such as diblock and triblock copolymers of polyoxyethylene and polyoxypropylene, including poloxamer 188, poloxamer 407, and poloxamer 338. Ionic surfactants such as sodium sulfosuccinate, and fatty acid soaps may also be utilized. Other lipids including glycolipids, ganglioside GM1, sphingomyelin, phosphatidic acid, cardiolipin; lipids bearing polymer chains such as polyethylene glycol, chitin, hyaluronic acid, or polyvinylpyrrolidone; lipids bearing sulfonated mono-, di-, and polysaccharides; fatty acids such as palmitic acid, stearic acid, and oleic acid; cholesterol, cholesterol esters, and cholesterol hemisuccinate may also be used in the pharmaceutical formulation when desirable. The optional additive may also comprise a biocompatible copolymer, or blend. In this respect potentially useful polymers comprise polylactides, polylactide-glycolides, cyclodextrins, polyacrylates, methylcellulose, carboxymethylcellulose, polyvinyl alcohols, polyanhydrides, polylactams, polyvinyl pyrrolidones, polysaccharides (dextrans, starches, chitin, chitosan, etc.), hyaluronic acid, proteins, (albumin, collagen, gelatin, etc.). Those skilled in the art will appreciate that, by selecting the appropriate polymers, the delivery efficiency of the particulate compositions and/or the stability of the dispersions may be tailored to optimize the effectiveness of the active or agent.

Alternatively or additionally, the pharmaceutical formulation may comprise an additive which is added to improve particle rigidity, production yield, emitted dose and deposition, shelf-life and patient acceptance. Such optional additives include, but are not limited to one or more of coloring agents, taste masking agents, buffers, hygroscopic agents, antioxidants, and chemical stabilizers. Furthermore, various excipients may be incorporated in, or added to, the particulate matrix to provide structure and form to the particulates. In this regard it will be appreciated that the rigidifying components can be removed using a post-production technique such as selective solvent extraction. Other excipients may include, but are not limited to, carbohydrates including monosaccharides, disaccharides and polysaccharides. For example, monosaccharides such as dextrose (anhydrous and monohydrate), galactose, mannitol, D-mannose, sorbitol, sorbose and the like; disaccharides such as lactose, maltose, sucrose, trehalose, and the like; trisaccharides such as raffinose and the like; and other carbohydrates such as starches (hydroxyethylstarch), cyclodextrins and maltodextrins. Other excipients suitable for use with the present invention, including amino acids, are known in the art such as those disclosed in WO 95/31479, WO 96/32096, and WO 96/32149. Mixtures of carbohydrates and amino acids are further held to be within the scope of the present invention. The inclusion of both inorganic (e.g. sodium chloride, etc.), organic acids and their salts (e.g. carboxylic acids and their salts such as sodium citrate, sodium ascorbate, magnesium gluconate, sodium gluconate, tromethamine hydrochloride, etc.) and buffers is also contemplated. The inclusion of salts and organic solids such as ammonium carbonate, ammonium acetate, ammonium chloride or camphor are also contemplated. Yet other potential additives include particulate compositions that may comprise, or may be coated with, charged species that prolong residence time at the point of contact or enhance penetration through mucosae. For example, anionic charges are known to favor mucoadhesion while cationic charges may be used to associate the formed microparticulate with negatively charged bioactive agents such as genetic material. The charges may be imparted through the association or incorporation of polyanionic or polycationic materials such as polyacrylic acids, polylysine, polylactic acid and chitosan.

In one version of the invention, the pharmaceutical formulation may comprise one or more targeting agents. For example, the pharmaceutical formulation may comprise a targeting agent that directs the spontaneously formed liposomes to cellular targets, such as pulmonary macrophages. This is particularly useful when the pharmaceutical formulation is being administered to treat an infectious disease where a pathogen is taken up by pulmonary macrophages. Such infectious diseases are difficult to treat with conventional systemic treatment with anti-infective active agents. However, by incorporating a targeting agent, the spontaneously formed liposome may be more readily taken up by the pulmonary macrophage and more effectively delivered to the site of infection. This method of treatment is particularly effective for the treatment of tuberculosis, bio-warfare agents, such as anthrax, and some types of cancer. The targeting agents may comprises, for example, one or more of phosphatidylserine, hIgG, and muramyl dipeptide, as described in PCT publications WO 99/06855, WO 01/64254, WO 02/09674, and WO 02/87542 and in U.S. Pat. No. 6,630,169, all of which are incorporated herein by reference in their entireties. The targeting process can be more effective if the active agent remains in the lungs for a long period of time. Accordingly, in one version, the pharmaceutical formulation comprises a targeting agent and sufficient amounts of the lipid component to encapsulate at least 70% of an active agent useful to treat an infectious disease where a pathogen is taken up by pulmonary macrophages. Particularly when the pharmaceutical formulation comprises such a targeting agent, the particle size is preferably less than 6 μm because larger particles are not readily taken up by pulmonary macrophages.

In one version, the hollow and/or porous microstructures discussed above are formed by spray drying, as disclosed in WO 99/16419. The spray drying process results in the formation of a particulate composition comprising particles having a relatively thin porous wall defining a large internal void. The spray drying process is also often advantageous over other processes in that the particles formed are less likely to rupture during processing or during deagglomeration. Spray drying is a one-step process that converts a liquid feed to a dried particulate form. The spray drying process involves bringing together a highly dispersed liquid, and a sufficient volume of hot air to produce evaporation and drying of the liquid droplets. The preparation to be spray dried or feedstock can be any solution, course suspension, slurry, colloidal dispersion, or paste that may be atomized using the selected spray drying apparatus. In preferred embodiments the feed stock will comprise a colloidal system such as an emulsion, reverse emulsion, microemulsion, multiple emulsion, particulate dispersion, or slurry. Typically the feed is sprayed into a current of warm filtered air that evaporates the solvent and conveys the dried product to a collector. The spent air is then exhausted with the solvent. Commercial spray dryers manufactured by Buchi Ltd. or Niro Corp. may be modified for use to produce the pharmaceutical formulation. Examples of spray drying methods and systems suitable for making the dry powders of the present invention are disclosed in U.S. Pat. Nos. 6,077,543, 6,051,256, 6,001,336, 5,985,248, and 5,976,574, all of which are incorporated herein by reference in their entireties.

In some instances dispersion stability and dispersibility of the spray dried particulate compositions can be improved by using a blowing agent, as described in WO 99/16419 cited above. This process forms an emulsion, optionally stabilized by an incorporated surfactant, typically comprising submicron droplets of water immiscible blowing agent dispersed in an aqueous continuous phase. The blowing agent may be a fluorinated compound (e.g. perfluorohexane, perfluorooctyl bromide, perfluorooctyl ethane, perfluorodecalin, perfluorobutyl ethane) which vaporizes during the spray-drying process, leaving behind generally hollow, porous aerodynamically light microspheres. Other suitable liquid blowing agents include nonfluorinated oils, chloroform, Freons, ethyl acetate, alcohols, hydrocarbons, nitrogen, and carbon dioxide gases.

Although the particulate compositions are preferably formed using a blowing agent as described above, it will be appreciated that, in some instances, no additional blowing agent is required and an aqueous dispersion of the medicament and/or excipients and surfactant(s) are spray dried directly. In such cases, the pharmaceutical formulation may possess special physicochemical properties (e.g., high crystallinity, elevated melting temperature, surface activity, etc.) that makes it particularly suitable for use in such techniques.

The first step in particulate production typically comprises feed stock preparation. If the phospholipid based particle is intended to act as a carrier for another active agent, the selected active agent is dissolved in a solvent, such as water, to produce a concentrated solution. The polyvalent cation may be added to the active agent solution or may be added to the phospholipid emulsion as discussed below. The active agent may also be dispersed directly in the emulsion, particularly in the case of water insoluble agents. Alternatively, the active agent may be incorporated in the form of a solid particulate dispersion. The concentration of the active agent used is dependent on the amount of agent required in the final powder and the performance of the delivery device employed. In one version, a polyvalent cation-containing oil-in-water emulsion is then formed in a separate vessel. The oil employed is preferably a fluorocarbon (e.g., perfluorooctyl bromide, perfluorooctyl ethane, perfluorodecalin) which is emulsified with a phospholipid. For example, polyvalent cation and phospholipid may be homogenized in hot distilled water (e.g., 60.degree. C.) using a suitable high shear mechanical mixer (e.g., Ultra-Turrax model T-25 mixer) at 8000 rpm for 2 to 5 minutes. Typically 5 to 25 g of fluorocarbon is added dropwise to the dispersed surfactant solution while mixing. The resulting polyvalent cation-containing perfluorocarbon in water emulsion is then processed using a high pressure homogenizer to reduce the particle size. Typically the emulsion is processed at 12,000 to 18,000 psi, 5 discrete passes and kept at 50 to 80.degree. C. The active agent solution and perfluorocarbon emulsion are then combined and fed into the spray dryer. Typically the two preparations will be miscible as the emulsion will preferably comprise an aqueous continuous phase. While the bioactive agent is solubilized separately for the purposes of the instant discussion it will be appreciated that, in other embodiments, the active agent may be solubilized (or dispersed) directly in the emulsion. In such cases, the active emulsion is spray dried without combining a separate active agent preparation.

Operating conditions such as inlet and outlet temperature, feed rate, atomization pressure, flow rate of the drying air, and nozzle configuration can be adjusted in order to produce the required particle size, and production yield of the resulting dry particles. Exemplary settings are as follows: an air inlet temperature between 60.degree. C. and 170.degree. C.; an air outlet between 40.degree. C. to 120.degree. C.; a feed rate between 3 ml to about 15 ml per minute; and an aspiration air flow of 300 L/min. and an atomization air flow rate between 25 to 50 L/min. The use of the described method provides for the formation of hollow and/or porous microstructures that are aerodynamically light microparticles with particle diameters appropriate for aerosol deposition into the lung, as discussed above.

Particulate compositions useful in the present invention may alternatively be formed by lyophilization. Lyophilization is a freeze-drying process in which water is sublimed from the composition after it is frozen. The particular advantage associated with the lyophilization process is that biologicals and pharmaceuticals that are relatively unstable in an aqueous solution can be dried without elevated temperatures, and then stored in a dry state where there are few stability problems. With respect to the instant invention such techniques are particularly compatible with the incorporation of peptides, proteins, genetic material and other natural and synthetic macromolecules in particulate compositions without compromising physiological activity. The lyophilized cake containing a fine foam-like structure can be micronized using techniques known in the art to provide the desired sized particles.

In one version, the pharmaceutical formulation is composed of hollow and/or porous microstructures having a bulk density less than 0.5 g/cm³, more preferably less than 0.3 g/cm³, and sometimes less 0.1 g/cm³. By providing particles with very low bulk density, the minimum powder mass that can be filled into a unit dose container is reduced, which eliminates the need for carrier particles. That is, the relatively low density of the powders of the present invention provides for the reproducible administration of relatively low dose pharmaceutical compounds. Moreover, the elimination of carrier particles will potentially minimize throat deposition and any “gag” effect, since the large lactose particles will impact the throat and upper airways due to their size.

The powder pharmaceutical formulation may be administered using an aerosolization device 105, as discussed above. The aerosolization device 105 may be a nebulizer, a metered dose inhaler, a liquid dose instillation device, or a dry powder inhaler. The powder pharmaceutical formulation 100 may be delivered by a nebulizer as described in WO 99/16420, by a metered dose inhaler as described in WO 99/16422, by a liquid dose instillation apparatus as described in WO 99/16421, and by a dry powder inhaler as described in U.S. patent application Ser. No. 09/888,311 filed on Jun. 22, 2001, in WO 02/83220, and in U.S. Pat. No. 6,546,929 all of these patents and patent applications being incorporated herein by reference in their entireties.

In one version, the pharmaceutical formulation 100 is in dry powder form and is contained within a unit dose receptacle which may be inserted into or near the aerosolization apparatus 105 to aerosolize the unit dose of the pharmaceutical formulation. This version is useful in that the dry powder form may be stably stored in its unit dose receptacle for a long period of time. In addition, this version is convenient in that no refrigeration or external power source is required for aerosolization.

In some instances, it is desirable to deliver high dose, such as doses greater than 10 mg of active agent to the lung in a single inhalation. The above described phospholipid hollow and/or porous microstructure based dry powder particulates allow for doses greater than 10 mg, sometimes greater than 25 mg, to be delivered in a single inhalation. To achieve this, the bulk density of the powder is preferably less than 0.4 g/cm³, and more preferably less than 0.2 g/cm³. Generally, a drug loading of more than 5% w/w, more preferably more than 10% w/w, more preferably more than 20% w/w, more preferably more than 30% w/w, and most preferably more than 40% w/w is also desirable when the required lung dose in more than 10 mg.

These high dose pharmaceutical formulations may be contained in a capsule that may be inserted into an aerosolization device 105. The capsule may be of a suitable shape, size, and material to contain the pharmaceutical formulation and to provide the pharmaceutical formulation in a usable condition. For example, the capsule may comprise a wall which comprises a material that does not adversely react with the pharmaceutical formulation. In addition, the wall may comprise a material that allows the capsule to be opened to allow the pharmaceutical formulation to be aerosolized. In one version, the wall comprises one or more of gelatin, hydroxypropyl methylcellulose (HPMC), polyethyleneglycol-compounded HPMC, hydroxyproplycellulose, agar, or the like. In one version, the capsule may comprise telescopically adjoining sections, as described for example in U.S. Pat. No. 4,247,066 which is incorporated herein by reference in its entirety. The size of the capsule may be selected to adequately contain the dose of the pharmaceutical formulation. The sizes generally range from size 5 to size 000 with the outer diameters ranging from about 4.91 mm to 9.97 mm, the heights ranging from about 11.10 mm to about 26.14 mm, and the volumes ranging from about 0.13 ml to about 1.37 ml, respectively. Suitable capsules are available commercially from, for example, Shionogi Qualicaps Co. in Nara, Japan and Capsugel in Greenwood, S.C. After filling, a top portion may be placed over the bottom portion to form the a capsule shape and to contain the powder within the capsule, as described in U.S. Pat. No. 4,846,876, U.S. Pat. No. 6,357,490, and in the PCT application WO 00/07572 published on Feb. 17, 2000, all of which are incorporated herein by reference in their entireties.

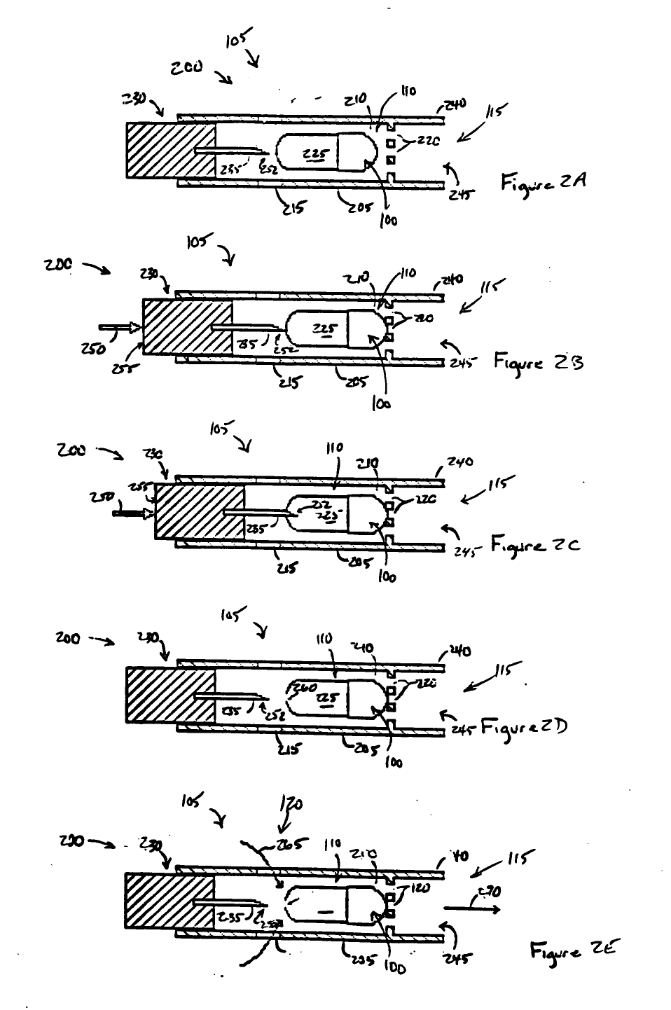

An example of a dry powder aerosolization apparatus 200 particularly useful in aerosolizing a pharmaceutical formulation 100 according to the present invention is shown schematically in FIG. 2A. The aerosolization apparatus 200 comprises a housing 205 defining a chamber 210 having one or more air inlets 215 and one or more air outlets 220. The chamber 210 is sized to receive a capsule 225 which contains an aerosolizable pharmaceutical formulation. A puncturing mechanism 230 comprises a puncture member 235 that is moveable within the chamber 210. Near or adjacent the outlet 220 is an end section 240 that may be sized and shaped to be received in a user's mouth or nose so that the user may inhale through an opening 245 in the end section 240 that is in communication with the outlet 220.

The dry powder aerosolization apparatus 200 utilizes air flowing through the chamber 210 to aerosolize the pharmaceutical formulation in the capsule 225. For example, FIGS. 2A through 2E illustrate the operation of a version of an aerosolization apparatus 200 where air flowing through the inlet 215 is used to aerosolize the pharmaceutical formulation and the aerosolized pharmaceutical formulation flows through the outlet 220 so that it may be delivered to the user through the opening 245 in the end section 240. The dry powder aerosolization apparatus 200 is shown in its initial condition in FIG. 2A. The capsule 225 is positioned within the chamber 210 and the pharmaceutical formulation is contained within the capsule 225.

To use the aerosolization apparatus 200, the pharmaceutical formulation in the capsule 225 is exposed to allow it to be aerosolized. In the version of FIGS. 2A though 2E, the puncture mechanism 230 is advanced within the chamber 210 by applying a force 250 to the puncture mechanism 230. For example, a user may press against a surface 255 of the puncturing mechanism 230 to cause the puncturing mechanism 230 to slide within the housing 205 so that the puncture member 235 contacts the capsule 225 in the chamber 210, as shown in FIG. 2B. By continuing to apply the force 250, the puncture member 235 is advanced into and through the wall of the capsule 225, as shown in FIG. 2C. The puncture member may comprise one or more sharpened tips 252 to facilitate the advancement through the wall of the capsule 225. The puncturing mechanism 230 is then retracted to the position shown in FIG. 2D, leaving an opening 260 through the wall of the capsule 225 to expose the pharmaceutical formulation in the capsule 225.

Air or other gas then flows through an inlet 215, as shown by arrows 265 in FIG. 2E. The flow of air causes the pharmaceutical formulation to be aerosolized. When the user inhales 270 through the end section 240 the aerosolized pharmaceutical formulation is delivered to the user's respiratory tract. In one version, the air flow 265 may be caused by the user's inhalation 270. In another version, compressed air or other gas may be ejected into the inlet 215 to cause the aerosolizing air flow 265.

A specific version of a dry powder aerosolization apparatus 200 is described in U.S. Pat. No. 4,069,819 and in U.S. Pat. No. 4,995,385, both of which are incorporated herein by reference in their entireties. In such an arrangement, the chamber 210 comprises a longitudinal axis that lies generally in the inhalation direction, and the capsule 225 is insertable lengthwise into the chamber 210 so that the capsule's longitudinal axis may be parallel to the longitudinal axis of the chamber 210. The chamber 210 is sized to receive a capsule 225 containing a pharmaceutical formulation in a manner which allows the capsule to move within the chamber 210. The inlets 215 comprise a plurality of tangentially oriented slots. When a user inhales through the endpiece, outside air is caused to flow through the tangential slots. This airflow creates a swirling airflow within the chamber 210. The swirling airflow causes the capsule 225 to contact a partition and then to move within the chamber 210 in a manner that causes the pharmaceutical formulation to exit the capsule 225 and become entrained within the swirling airflow. This version is particularly effective in consistently aerosolizing high doses if the pharmaceutical formulation. In one version, the capsule 225 rotates within the chamber 210 in a manner where the longitudinal axis of the capsule is remains at an angle less than 80 degrees, and preferably less than 45 degrees from the longitudinal axis of the chamber. The movement of the capsule 225 in the chamber 210 may be caused by the width of the chamber 210 being less than the length of the capsule 225. In one specific version, the chamber 210 comprises a tapered section that terminates at an edge. During the flow of swirling air in the chamber 210, the forward end of the capsule 225 contacts and rests on the partition and a sidewall of the capsule 225 contacts the edge and slides and/or rotates along the edge. This motion of the capsule is particularly effective in forcing a large amount of the pharmaceutical formulation through one or more openings 260 in the rear of the capsule 225.

In another version, the dry powder aerosolization apparatus 200 may be configured differently than as shown in FIGS. 2A through 2E. For example, the chamber 210 may be sized and shaped to receive the capsule 225 so that the capsule 225 is orthogonal to the inhalation direction, as described in U.S. Pat. No. 3,991,761. As also described in U.S. Pat. No. 3,991,761, the puncturing mechanism 230 may puncture both ends of the capsule 225. In another version, the chamber may receive the capsule 225 in a manner where air flows through the capsule 225 as described for example in U.S. Pat. No. 4,338,931 and in U.S. Pat. No. 5,619,985. In another version, the aerosolization of the pharmaceutical formulation may be accomplished by pressurized gas flowing through the inlets, as described for example in U.S. Pat. No. 5,458,135, U.S. Pat. No. 5,785,049, and U.S. Pat. No,. 6,257,233, or propellant, as described in PCT Publication WO 00/72904 and U.S. Pat. No. 4,114,615. All of the above references being incorporated herein by reference in their entireties.

The active agent described herein includes an agent, drug compound, composition of matter or mixture thereof which provides some pharmacologic, often beneficial, effect. This includes foods, food supplements, nutrients, drugs, vaccines, vitamins, and other beneficial agents. As used herein, the terms further include any physiologically or pharmacologically active substance that produces a localized or systemic effect in a patient. An active agent for incorporation in the pharmaceutical formulation described herein may be an inorganic or an organic compound, including, without limitation, drugs which act on: the peripheral nerves, adrenergic receptors, cholinergic receptors, the skeletal muscles, the cardiovascular system, smooth muscles, the blood circulatory system, synoptic sites, neuroeffector junctional sites, endocrine and hormone systems, the immunological system, the reproductive system, the skeletal system, autacoid systems, the alimentary and excretory systems, the histamine system, and the central nervous system. Suitable active agents may be selected from, for example, hypnotics and sedatives, psychic energizers, tranquilizers, respiratory drugs, anticonvulsants, muscle relaxants, antiparkinson agents (dopamine antagonists), analgesics, anti-inflammatories, antianxiety drugs (anxiolytics), appetite suppressants, antimigraine agents, muscle contractants, anti-infectives (antibiotics, antivirals, antifungals, vaccines) antiarthritics, antimalarials, antiemetics, anepileptics, bronchodilators, cytokines, growth factors, anti-cancer agents, antithrombotic agents, antihypertensives, cardiovascular drugs, antiarrhythmics, antioxicants, anti-asthma agents, hormonal agents including contraceptives, sympathomimetics, diuretics, lipid regulating agents, antiandrogenic agents, antiparasitics, anticoagulants, neoplastics, antineoplastics, hypoglycemics, nutritional agents and supplements, growth supplements, antienteritis agents, vaccines, antibodies, diagnostic agents, and contrasting agents. The active agent, when administered by inhalation, may act locally or systemically.

The active agent may fall into one of a number of structural classes, including but not limited to small molecules, peptides, polypeptides, proteins, polysaccharides, steroids, proteins capable of eliciting physiological effects, nucleotides, oligonucleotides, polynucleotides, fats, electrolytes, and the like.

Examples of active agents suitable for use in this invention include but are not limited to one or more of calcitonin, erythropoietin (EPO), Factor VIII, Factor IX, ceredase, cerezyme, cyclosporin, granulocyte colony stimulating factor (GCSF), thrombopoietin (TPO), alpha-1 proteinase inhibitor, elcatonin, granulocyte macrophage colony stimulating factor (GMCSF), growth hormone, human growth hormone (HGH), growth hormone releasing hormone (GHRH), heparin, low molecular weight heparin (LMWH), interferon alpha, interferon beta, interferon gamma, interleukin-1 receptor, interleukin-2, interleukin-1 receptor antagonist, interleukin-3, interleukin-4, interleukin-6, luteinizing hormone releasing hormone (LHRH), factor IX, insulin, pro-insulin, insulin analogues (e.g., mono-acylated insulin as described in U.S. Pat. No. 5,922,675, which is incorporated herein by reference in its entirety), amylin, C-peptide, somatostatin, somatostatin analogs including octreotide, vasopressin, follicle stimulating hormone (FSH), insulin-like growth factor (IGF), insulintropin, macrophage colony stimulating factor (M-CSF), nerve growth factor (NGF), tissue growth factors, keratinocyte growth factor (KGF), glial growth factor (GGF), tumor necrosis factor (TNF), endothelial growth factors, parathyroid hormone (PTH), glucagon-like peptide thymosin alpha 1, IIb/IIIa inhibitor, alpha-1 antitrypsin, phosphodiesterase (PDE) compounds, VLA-4 inhibitors, bisphosponates, respiratory syncytial virus antibody, cystic fibrosis transmembrane regulator (CFTR) gene, deoxyreibonuclease (Dnase), bactericidal/permeability increasing protein (BPI), anti-CMV antibody, 13-cis retinoic acid, macrolides such as erythromycin, oleandomycin, troleandomycin, roxithromycin, clarithromycin, davercin, azithromycin, flurithromycin, dirithromycin, josamycin, spiromycin, midecamycin, leucomycin, miocamycin, rokitamycin, andazithromycin, and swinolide A; fluoroquinolones such as ciprofloxacin, ofloxacin, levofloxacin, trovafloxacin, alatrofloxacin, moxifloxicin, norfloxacin, enoxacin, grepafloxacin, gatifloxacin, lomefloxacin, sparfloxacin, temafloxacin, pefloxacin, amifloxacin, fleroxacin, tosufloxacin, prulifloxacin, irloxacin, pazufloxacin, clinafloxacin, and sitafloxacin, aminoglycosides such as gentamicin, netilmicin, paramecin, tobramycin, amikacin, kanamycin, neomycin, and streptomycin, vancomycin, teicoplanin, rampolanin, mideplanin, colistin, daptomycin, gramicidin, colistimethate, polymixins such as polymixin B, capreomycin, bacitracin, penems; penicillins including penicillinase-sensitive agents like penicillin G, penicillin V, penicillinase-resistant agents like methicillin, oxacillin, cloxacillin, dicloxacillin, floxacillin, nafcillin; gram negative microorganism active agents like ampicillin, amoxicillin, and hetacillin, cillin, and galampicillin; antipseudomonal penicillins like carbenicillin, ticarcillin, azlocillin, mezlocillin, and piperacillin; cephalosporins like cefpodoxime, cefprozil, ceftbuten, ceftizoxime, ceftriaxone, cephalothin, cephapirin, cephalexin, cephradrine, cefoxitin, cefamandole, cefazolin, cephaloridine, cefaclor, cefadroxil, cephaloglycin, cefuroxime, ceforanide, cefotaxime, cefatrizine, cephacetrile, cefepime, cefixime, cefonicid, cefoperazone, cefotetan, cefmetazole, ceftazidime, loracarbef, and moxalactam, monobactams like aztreonam; and carbapenems such as imipenem, meropenem, pentamidine isethiouate, albuterol sulfate, lidocaine, metaproterenol sulfate, beclomethasone diprepionate, triamcinolone acetamide, budesonide acetonide, fluticasone, ipratropium bromide, flunisolide, cromolyn sodium, ergotamine tartrate and where applicable, analogues, agonists, antagonists, inhibitors, and pharmaceutically acceptable salt forms of the above. In reference to peptides and proteins, the invention is intended to encompass synthetic, native, glycosylated, unglycosylated, pegylated forms, and biologically active fragments and analogs thereof.

Active agents for use in the invention further include nucleic acids, as bare nucleic acid molecules, vectors, associated viral particles, plasmid DNA or RNA or other nucleic acid constructions of a type suitable for transfection or transformation of cells, i.e., suitable for gene therapy including antisense. Further, an active agent may comprise live attenuated or killed viruses suitable for use as vaccines. Other useful drugs include those listed within the Physician's Desk Reference (most recent edition).

The amount of active agent in the pharmaceutical formulation will be that amount necessary to deliver a therapeutically effective amount of the active agent per unit dose to achieve the desired result. In practice, this will vary widely depending upon the particular agent, its activity, the severity of the condition to be treated, the patient population, dosing requirements, and the desired therapeutic effect. The composition will generally contain anywhere from about 1% by weight to about 99% by weight active agent, typically from about 2% to about 95% by weight active agent, and more typically from about 5% to 85% by weight active agent, and will also depend upon the relative amounts of additives contained in the composition. The compositions of the invention are particularly useful for active agents that are delivered in doses of from 0.001 mg/day to 100 mg/day, preferably in doses from 0.01 mg/day to 75 mg/day, and more preferably in doses from 0.10 mg/day to 50 mg/day. It is to be understood that more than one active agent may be incorporated into the formulations described herein and that the use of the term “agent” in no way excludes the use of two or more such agents.

### EXAMPLE 1

In one example, the pharmaceutical formulation 100 of the present invention comprises an anti-infective active agent, such as ciprofloxacin. Ciprofloxacin, various forms of which are described in U.S. Pat. 4,670,444 which is incorporated herein by reference in its entirety, is useful in treating infections of in the lungs, such as cystic fibrosis, gram negative infections such as pseudomonas aeruginosa, bronchiectasis, COPD, and chronic bronchitis. Aerosolized ciprofloxacin, when administered to the lungs, has a very short half life in the lungs. Therefore, by spontaneously encapsulating the ciprofloxacin in a liposome as described above, the retention of the ciprofloxacin in the lungs is extended and the effectiveness of the active agent is increased.

In one useful version of the invention, the pharmaceutical formulation may comprise ciprofloxacin for the purpose of treating a person who has been exposed to inhalation anthrax infection or a person who is in danger of coming into contact with inhalation anthrax. For example, the pharmaceutical formulation may be administered to soldiers, to postal workers, or to others who have been or may be exposed to anthrax spores.

Endospores of Bacillus anthracis are about 1-2 mm in diameter, optimal for deposition into the deep lung. Endospores are generally phagocytosed by pulmonary macrophages and cleared to mediastinal and peribronchial lymph nodes, where the endospores germinate and release bacilli inside the macrophages. While incubation times are on the order of 10 days, symptoms may occur up to 6 weeks following inhalation, reflecting the ability of endospores to remain in the lungs for extended periods of time. Anthrax bacilli multiply in the lymph nodes, causing hemorrhagic mediastinitis. Eventually the bacteria enter the bloodstream via the thoracic duct, resulting in severe septicemia and often death. Once endospores are cleared to the regional lymph nodes, oral or parenteral treatment with anti-infectives is less efficacious. Local lung delivery allows higher doses of anti-infective, such as the ciprofloxacin to be delivered to the lungs, without correspondingly higher systemic levels, thereby improving the therapeutic index. And most importantly, administration via inhalation is the only way to effectively target the therapeutic to the actual site of the anthrax infection.

By administering a pharmaceutical formulation comprising an ant-iinfective, a lipid component with desired liquid transition temperature characteristics and with a targeting agent, as described above, the inhalation anthrax can be treated. Ciprofloxacin is currently the anti-infective of choice for treating pulmonary infections of B. anthracis. Ciprofloxacin is a potent and broad-spectrum fluoroquinolone that is especially effective against gram negative pathogens. It is also effective against several pathogens that cause respiratory infections (e.g., Mycobacterium tuberculosis, Mycobacterium avium-M. intracellulare, Hemophilus influenzae, and Pseudomonas aeruginosa).

In one version, high doses of a pharmaceutical formulation as described above and comprising ciprofloxacin may be stored in a capsule and administered in a dry powder aerosolization apparatus, as shown in FIGS. 2A through 2E. Accordingly, the equipment may be 5 easily carried as part of a soldier's military equipment and may be easily stored in a hospital or a postal facility.

### EXAMPLE 2

In another example, the pharmaceutical formulation 100 of the present invention can be used to treat mycobacterium, such as tuberculosis. Accordingly, in this version, the pharmaceutical formulation comprises an anti-tuberculosis agent, such as rifampin and/or isoniazid. Since mycobacterium infections are subject to uptake by pulmonary macrophages, it is preferable for the pharmaceutical formulation according to this version to also comprise a targeting agent, as described above.

### EXAMPLE 3

In another example, the pharmaceutical formulation 100 of the present invention can be used to treat cancer. Accordingly, in this version, the pharmaceutical formulation comprises an oncolytic agent, such as one or more of doxorubicin, platinol, paclitaxel, fluorouracil, cytarabine, 9-aminocamptothecin, cyclophosphamide, carboplatin, etoposide, bleomycin, vincristine, vinorelbine, mitomycin-C, and their associated classes and equivalents. Since the uptake of the active agent by pulmonary macrophages may deliver the active agent to the cite of some cancers, it may be preferable in some instances for the pharmaceutical formulation according to this version to also comprise a targeting agent, as described above.

### EXAMPLE 4

In another example, the pharmaceutical formulation 100 of the invention may comprise an active agent for which there is a desire for increasing the active agent's residence time in the lungs. For example, the active agent may comprise one or more asthma agents, such as formoterol and budesonide.

### EXAMPLE 5

In another example, the pharmaceutical formulation 100 may comprise an active agent useful in treating pulmonary Mycobacterium avium-intracellulare (MAI) infections. In this version, a pharmaceutical formulation comprising an anti-mycobacterial agent may be administered in a dose of at least 10 mg. The anti-mycobacterial agent is spontaneously encapsulated in the lungs when the pharmaceutical formulation is administered to the lungs.

### EXAMPLE 6

In another example, the pharmaceutical formulation 100 may comprise an active agent useful in treating pulmonary aspergilossis and other fungal infections. In this version, a pharmaceutical formulation comprising an anti-fungal agent, such as Amphotericin B, may be administered in a dose of at least 5 mg. The anti-fungal agent is spontaneously encapsulated in the lungs when the pharmaceutical formulation is administered to the lungs.

### EXAMPLE 7

In another example, the pharmaceutical formulation 100 may comprise an active agent useful in treating diseases that infect monocytes and macrophages, such as Listeria, Brucella, Leishmania and Mycobacterium avium-intracellulare. Accordingly, in this version, the pharmaceutical formulation comprises an anti-infective agent, such as amikacin, Since mycobacterium infections are subject to uptake by pulmonary macrophages, it is preferable for the pharmaceutical formulation according to this version to also comprise a targeting agent, as described above.

### EXAMPLE 8

In another example, the pharmaceutical formulation 100 may comprise an active agent useful in treating Pseudomonas aeruginosa (PA) infections. In this version, a pharmaceutical formulation comprising an anti-infective agent may be administered in a dose of at least 5 mg. The anti-infective agent is spontaneously encapsulated in the lungs when the pharmaceutical formulation is administered to the lungs.

Although the present invention has been described in considerable detail with regard to certain preferred versions thereof, other versions are possible, and alterations, permutations and equivalents of the version shown will become apparent to those skilled in the art upon a reading of the specification and study of the drawings. For example, the cooperating components may be reversed or provided in additional or fewer number. Also, the various features of the versions herein can be combined in various ways to provide additional versions of the present invention. Furthermore, certain terminology has been used for the purposes of descriptive clarity, and not to limit the present invention. Therefore, any appended claims should not be limited to the description of the preferred versions contained herein and should include all such alterations, permutations, and equivalents as fall within the true spirit and scope of the present invention.

1. A pharmaceutical formulation for pulmonary administration, the pharmaceutical formulation comprising:
a lipid component; and
an active agent;
wherein the pharmaceutical formulation has a liquid phase transition temperature of less than or equal to 37° C. when hydrated and a liquid phase transition temperature of greater than 57° C. when non-hydrated, whereby the lipid component spontaneously encapsulates and/or entraps the active agent when the pharmaceutical formulation is administered to the lungs.

2. A pharmaceutical formulation according to claim 1 wherein the pharmaceutical formulation has a liquid phase transition temperature greater than 67° C. when non-hydrated.

3. A pharmaceutical formulation according to claim 1 wherein the pharmaceutical formulation has a liquid phase transition temperature greater than 77° C. when non-hydrated.

4. A pharmaceutical formulation according to claim 1 wherein at least 50% of the active agent is spontaneously encapsulated or entrapped within the lipid.

5. A pharmaceutical formulation according to claim 1 wherein at least 70% of the active agent is spontaneously encapsulated within the lipid.

6. A pharmaceutical formulation according to claim 1 wherein the lipid component comprises one or more phospholipids.

7. A pharmaceutical formulation according to claim 6 wherein the lipid component comprises dimyristoylphosphatidylcholine.

8. A pharmaceutical formulation according to claim 6 wherein the lipid component comprises dilaurylphosphatidylcholine.

9. A pharmaceutical formulation according to claim 6 wherein the lipid component comprises dipalmitoylphosphatidylcholine.

10. A pharmaceutical formulation according to claim 6 wherein the lipid component comprises a mixture of dimyristoylphosphatidylcholine and dipalmitoylphosphatidylcholine.

11. A pharmaceutical formulation according to claim 6 wherein the lipid component comprises a mixture of one or more uncharged phospholipids and one or more charged phospholipids.

12. A pharmaceutical formulation according to claim 1 wherein the lipid component comprises a first component having a liquid phase transition temperature of greater than 37° C. and a second component having a liquid phase transition temperature of less than 37° C.

13. A pharmaceutical formulation according to claim 1 wherein the pharmaceutical formulation further comprises divalent cations.

14. A pharmaceutical formulation according to claim 13 wherein the divalent cations are present in an amount sufficient to raise the liquid transition temperature of the pharmaceutical formulation when non-hydrated.

15. A pharmaceutical formulation according to claim 13 wherein the divalent cations are calcium ions.

16. A pharmaceutical formulation according to claim 1 wherein the pharmaceutical formulation further comprises a targeting agent.

17. A pharmaceutical formulation according to claim 16 wherein the targeting agent is an agent which will direct the spontaneously encapsulated active agent to pulmonary macrophages.

18. A pharmaceutical formulation according to claim 16 wherein the targeting agent comprises one or more agents from the group consisting of phosphatidylserine, hIgG, muramyl dipeptide, and N-acetyl cysteine.

19. A pharmaceutical formulation according to claim 1 wherein the pharmaceutical formulation comprises a dry powder having a bulk density less than 0.5 g/cm³.

20. A pharmaceutical formulation according to claim 1 wherein the pharmaceutical formulation comprises a dry powder having a bulk density less than 0.4 g/cm³.

21. A pharmaceutical formulation according to claim 1 wherein the pharmaceutical formulation comprises a dry powder having a bulk density less than 0.2 g/cm³.

22. A pharmaceutical formulation according to claim 1 substantially absent large carrier particles.

23. A pharmaceutical formulation according to claim 1 substantially absent large carrier particles comprising lactose.

24. A pharmaceutical formulation according to claim 1 wherein the pharmaceutical formulation comprises a dry powder comprising particles having a median geometric diameter, as determined by laser diffraction, of less than 20 μm.

25. A pharmaceutical formulation according to claim 1 wherein the pharmaceutical formulation comprises a dry powder comprising particles having a median geometric diameter, as determined by laser diffraction, of less than 6 μm.

26. A pharmaceutical formulation according to claim 1 wherein the pharmaceutical formulation comprises a dry powder comprising particles, wherein each particles comprises a lipid component and an active agent.

27. A pharmaceutical formulation according to claim 24 wherein the particles are hollow or porous.

28. A pharmaceutical formulation according to claim 1 wherein a unit dose of the pharmaceutical formulation is contained within a receptacle.

29. A pharmaceutical formulation according to claim 26 wherein at least 10 mg of the pharmaceutical formulation is contained within the receptacle.

30. A pharmaceutical formulation according to claim 26 wherein at least 25 mg of the pharmaceutical formulation is contained within the receptacle.

31. A pharmaceutical formulation according to claim 26 wherein the receptacle is a capsule that may be inserted into a capsule-based dry powder inhaler.

32. A pharmaceutical formulation according to claim 1 wherein the active agent comprises an anti-infective.

33. A pharmaceutical formulation according to claim 32 wherein the anti-infective is selected to treat an inhalational anthrax infection.

34. A pharmaceutical formulation according to claim 32 wherein the anti-infective is selected to treat tuberculosis.

35. A pharmaceutical formulation according to claim 1 wherein the active agent comprises ciprofloxacin.

36. A pharmaceutical formulation according to claim 35 wherein the ciprofloxacin is ciprofloxacin HCl.

37. A pharmaceutical formulation according to claim 1 wherein the active agent comprises an anti-cancer therapeutic.

38. A pharmaceutical formulation according to claim 1 wherein the active agent comprises an anti asthma agent therapeutic.

39. A pharmaceutical formulation according to claim 38 wherein the active agent comprises one or more of formoterol and budesonide.

40. A pharmaceutical formulation for pulmonary administration, the pharmaceutical formulation comprising:
a lipid component;
ciprofloxacin; and
a targeting agent,
wherein the pharmaceutical formulation has a liquid phase transition temperature of less than or equal to 37° C. when hydrated, whereby the lipid component spontaneously encapsulates and/or entraps the active agent when the pharmaceutical formulation is administered to the lungs.

41. A method of administering an active agent to the lungs of a user, the method comprising:
providing a receptacle containing a dry powder pharmaceutical formulation comprising a lipid component and an active agent;
aerosolizing the pharmaceutical formulation and administering the pharmaceutical formulation to the lungs of the user during the user's inhalation; and
spontaneously encapsulating or entrapping the active agent within the lipid when the pharmaceutical formulation enters the lungs.

42. A method according to claim 41 wherein the pharmaceutical formulation has a liquid phase transition temperature of less than or equal to 37° C. when hydrated, whereby the lipid component spontaneously encapsulates and/or entraps the active agent when the pharmaceutical formulation is administered to the lungs.

43. A method according to claim 41 wherein the active agent comprises ciprofloxacin.

44. A method according to claim 42 wherein the pharmaceutical formulation is administered to the user after the user has been exposed to inhalation anthrax.

45. A method according to claim 42 wherein the pharmaceutical formulation is administered to the user prophylacticly before the user has been exposed to inhalation anthrax.

46. A method according to claim 41 wherein at least 5 mg of the pharmaceutical formulation is administered to the user during the users inhalation.

47. A method according to claim 41 wherein the active agent comprises an anti-infective agent and wherein the pharmaceutical formulation is administered to a user having a pulmonary infection.

48. A method according to claim 41 wherein the active agent comprises an anti-infective agent and wherein the pharmaceutical formulation is administered to a user having cystic fibrosis.

49. A method according to claim 41 wherein the active agent comprises an anti-fungal agent and wherein the pharmaceutical formulation is administered to a user having a pulmonary fungal infection.

50. A method according to claim 41 wherein the active agent comprises Amphotericin B and wherein the pharmaceutical formulation is administered to a user having a pulmonary fungal infection.

51. A method according to claim 41 wherein the active agent comprises an anti-fungal agent and wherein the pharmaceutical formulation is administered to a user prophylacticly.

52. A method according to claim 41 wherein the active agent comprises an anti-tuberculosis agent and wherein the pharmaceutical formulation is administered to a user having a tuberculosis.

53. A method according to claim 41 wherein the active agent comprises an anti-cancer agent and wherein the pharmaceutical formulation is administered to a user having lung cancer.

54. A method according to claim 41 wherein the active agent comprises an anti-asthma agent and wherein the pharmaceutical formulation is administered to a user having asthma.

55. A pharmaceutical formulation for pulmonary administration, the pharmaceutical formulation comprising:
a lipid component; and
ciprofloxacin;
wherein the pharmaceutical formulation has a liquid phase transition temperature of less than or equal to 37° C. when hydrated, whereby the lipid component spontaneously encapsulates and/or entraps the active agent when the pharmaceutical formulation is administered to the lungs.

56. A pharmaceutical formulation according to claim 55 wherein the ciprofloxacin comprises ciprofloxacin HCl.

57. A pharmaceutical formulation for pulmonary administration, the pharmaceutical formulation comprising:
a lipid component;
an active agent; and
a targeting agent,
wherein the pharmaceutical formulation has a liquid phase transition temperature of less than or equal to 37° C. when hydrated, whereby the lipid component spontaneously encapsulates and/or entraps the active agent when the pharmaceutical formulation is administered to the lungs.

58. A pharmaceutical formulation according to claim 57 wherein the targeting agent comprises one or more agents from the group consisting of phosphatidylserine, hIgG, muramyl dipeptide, and N-acetyl cysteine.
